(19) United States
(12) Patent Application Publication
(10) Pub. No.: US 2014/0260528 A1
(43) Pub. Date: Sep. 18, 2014

(54) DETECTING CONTAMINATION

(71) Applicant: Martin Schoell, Berkeley, CA (US)

(72) Inventor: Martin Schoell, Berkeley, CA (US)

(21) Appl. No.: 13/841,522

(22) Filed: Mar. 15, 2013

(51) Int. Cl. G01N 33/00; G06F 19/00

(52) U.S. Cl. CPC G01N 33/0004; G06F 19/708
USPC 73/19.01; 73/23.2

(57) ABSTRACT

Methods, systems, and apparatus, including computer programs encoded on computer storage media, for detecting gas seepage. One of the methods includes obtaining surface gas sensor measurements from each of a plurality of locations within a geographic area prior to shale-gas development; determining whether natural gas seepage is occurring and establishing a corresponding baseline threshold value; obtaining additional surface gas sensor measurements after shale-gas development; determining that the additional gas sensor measurements for one or more of the plurality of locations exceed the baseline threshold level; performing field analysis of the gas sensor measurements that were determined to exceed the specified threshold to determine gas provenance, wherein the analysis distinguishes between natural gas seepage and thermogenic leakage; and presenting data indicating a geographic extent of gas thermogenic leakage based on the analysis.

### BACKGROUND

This specification relates to oil and gas production.

Oil and gas production has been increasing in recent years, particularly with respect to gas bearing formations such as shale gas formations. One technique for developing gas bearing formations is hydraulic fracturing. However, hydraulic fracturing has also been associated with concerns about potential environmental impacts including groundwater contamination.

### SUMMARY

Systems and methods are provided for monitoring and analyzing gas sensor data to determine contamination associated with shale-gas gas production. Surface gas sensors positioned within a geographic area relative to a well site can be used initially to identify methane leaks and their origin. When methane leakage is detected within a geographic region, the origin of the leakage can be determined. While naturally occurring seepage can exist independent of shale-gas production, detected methane can be analyzed to determine whether gas leakage resulting from shale-gas production is occurring. When such leakage is determined, groundwater analysis can be performed to identify hydrocarbon contamination, for example, of benzene, toluene, and xylene. The methane leakage and groundwater contamination data can be stored as well as presented, either separately or together, in one or more plots. The plots can provide visual representations of the geographic extent of contamination as well as the relative concentrations.

In general, one innovative aspect of the subject matter described in this specification can be embodied in methods that include the actions of obtaining surface gas sensor measurements from each of multiple locations within a geographic area prior to shale-gas development; determining whether natural gas seepage is occurring and establishing a corresponding baseline threshold value; obtaining additional surface gas sensor measurements after shale-gas development; determining that the additional gas sensor measurements for one or more of the multiple locations exceed the baseline threshold level; performing field analysis of the gas sensor measurements that were determined to exceed the specified threshold to determine gas provenance, wherein the analysis distinguishes between natural gas seepage and thermogenic leakage; and presenting data indicating a geographic extent of gas thermogenic leakage based on the analysis.

The foregoing and other embodiments can each optionally include one or more of the following features, alone or in combination. The method includes: obtaining isotope data for methane identified in the analysis as having thermogenic provenance. The method includes: comparing the obtained isotope data with additional isotope data obtained from gases removed during hydraulic fracturing of a horizontal well. The method includes: obtaining groundwater samples at locations corresponding to locations identified as having thermogenic provenance; and analyzing the groundwater for evidence of hydrocarbon contamination. The method includes: providing data visualizing a geographic extent of hydrocarbon contamination. Visualizing the geographic extent includes generating one or more of a map indicating a geographic region of hydrocarbon contamination, a three dimensional plot of hydrocarbon concentration with respect to location coordinates, or a scatterplot of hydrocarbon concentration with respect to location coordinates. Analyzing the groundwater data includes measuring levels of BTEX. Presenting data indicating a geographical extent of gas leakage includes generating a map representation indicating the geographical extent of the gas leakage. Generating the map representation includes generating a three-dimensional plot indicating a concentration of gas leakage with respect to geographic location. Generating the map representation includes generating a scatter plot indicating a concentration of gas leakage with respect to geographic location. Obtaining gas sensor measurements includes receiving data collected from multiple gas sensors, each gas sensor being positioned at a particular location within the geographic region. The gas sensors include one or more of a gas flux meter, a laser sensor, or an infrared Fourier transform infrared spectrometer. Determining gas origin includes performing isotope analysis. The data is presented relative to hydraulic fracturing operations. The method further includes: determining whether to perform hydrocarbon analysis; obtaining hydrocarbon data from groundwater samples associated with locations identified in the analysis as having a gas measurements of thermogenic provenance that exceed the threshold concentration; and performing isotopic analysis of the hydrocarbon data for evidence of hydrocarbon contamination.

In general, one innovative aspect of the subject matter described in this specification can be embodied in systems that include multiple surface gas sensors, each surface gas sensor configure to collect data associated with measurements of one or more of methane or particular hydrocarbon concentrations; one or more computers configured to perform operations on the data from the multiple gas sensors to determine a geographic extent of gas contamination and to generate visual representations of the data.

In general, one innovative aspect of the subject matter described in this specification can be embodied in methods that include the actions of obtaining gas sensor measurements from each of multiple locations within a geographic area, wherein the gas sensors are surface sensors measuring gas emitted from the surface; determining that the gas sensor measurements for one or more of the multiple locations indicates gas leakage; analyzing groundwater samples associated with locations in which the gas sensor measurements indicated gas leakage; and presenting data indicating a geographic extent of hydrocarbon contamination based on the analysis.

Particular embodiments of the subject matter described in this specification can be implemented so as to realize one or more of the following advantages. Methane leakage and/or groundwater contamination from shale-gas production can be rapidly identified directly in the field during and after drilling operations. Furthermore, the origin of gas leakage can be determined so that leakage from shale-gas production can be distinguished from naturally occurring seepage. As a result, the impact of escaped gas from production techniques such as hydraulic fracturing can be rapidly assessed.

Surface gas measurements can be used to identify methane leakage. Groundwater analysis can be performed more efficiently by targeting those geographic areas in which gas-production based leakage is detected.

Initial measurements can be performed, for example for naturally occurring methane seepage prior to shale-gas production. If natural seepage is detected, it provides a baseline for future gas measurements. Subsequent measurements can be taken after hydraulic fracturing and/or during production to monitor for changes. If the levels of future gas measurements exceed threshold values, further analysis is performed to determine whether non-natural leakage is occurring. If non-natural leakage is detected, additional analysis such as hydrocarbon analysis of groundwater can be performed.

Results can be visualized to provide a greater understanding of the lateral extent and levels of gas leakage and/or groundwater contamination, for example, relative to one or more gas production wells.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a cross-sectional diagram of an example of shale gas development using hydraulic fracturing.

FIG. 2 is a cross-sectional diagram of an example of gas contamination.

FIG. 3 is a diagram of an example sensor distribution for detecting gas contamination over a geographic area.

FIG. 4 is a flow diagram of an example process for detecting gas leakage.

FIG. 5 is a diagram of an example area of contamination relative to sensor measurements.

FIG. 6 is a three-dimensional plot showing an example of methane gas concentration with respect to geographic location.

Like reference numbers and designations in the various drawings indicate like elements.

### DETAILED DESCRIPTION

FIG. 1 is a cross-sectional diagram 100 of an example of shale gas development using hydraulic fracturing. A gas well 102 includes vertical and horizontal wellbore portions 104 that extend into a gas-bearing formation 106. The gas-bearing formation can include, for example, so-called “unconventional reservoirs” such as shale rock or coal beds. The horizontal wellbore portions allow for greater exposure to the gas bearing formation 106 than a conventional vertical wellbore.

Hydraulic fracturing is used to create conductive fractures in the gas bearing formation 106 to extract gas from shale reservoirs. Hydraulic fracturing enables the production of natural gas and oil from rock formations deep below the earth's surface e.g., 5,000-10,000 feet.

A hydraulic fracture is formed by pumping a fracturing fluid into the wellbore at a rate sufficient to increase pressure within the horizontal wellbore to form rock cracks in the gas-bearing formation 106, thereby releasing gas.

FIG. 2 is a cross-sectional diagram 200 of an example of gas contamination. In particular, diagram 200 illustrates potential escape pathways of gas, e.g., methane or hydrocarbons, due to hydraulic fracturing of gas-bearing formations. The cross-sectional diagram 200 illustrates several layers of strata below the earth's surface from shale gas development 202 to the ground surface 204. Gas can escape, e.g., leak, due to fracturing techniques through faults or other fractured rocks 206. The escaping gas can include methane and hydrocarbons e.g., BTEX.

The escaping gas can reach a water table 208, which can lead to groundwater contamination 210. Gas can further escape beyond the water table, e.g., through additional fractures, veins, or other pathways, that can result in microseepage 212 under the ground surface 204. The gas can further escape through the ground surface 204 into the atmosphere 214. Gas escaping at the ground surface 204 into the atmosphere 214 can be measured, for example, by measuring gas flux 216 or gas concentration, depending on the sensors used.

FIG. 3 is a diagram 300 of an example sensor distribution for detecting gas contamination over a geographic area 301. FIG. 3 shows a top view of the geographic area 301 surrounding a gas well 302. The gas well includes a horizontal wellbore 304 deep below the surface and having a path illustrated by the dashed line. A number of gas sensors 306 can be positioned on the surface relative to the well, and in particular, in correspondence with the hydraulic fracturing zone of the horizontal wellbore. The gas sensors 306 are modern instruments, selected on the basis of wide tests and field validations, for the detection of natural gas seepage. A TDLAS (Tunable Diode Laser Absorption Spectroscopy) sensor will measure methane (with accuracy 0.1 ppmv (parts per million by volume) and a lower detection limit of 0.1 ppmv); an infrared sensor will measure carbon dioxide (accuracy 2%, repeatability±5 ppmv); a FTIR sensor (Fourier Transform Infrared spectrometer) will measure all gaseous hydrocarbons and BTEX (Benzene, Toluene, Ethylbenzene and Xylenes). All these sensors can be associated to a small soil probe (max 1 meter deep) for gas concentration measurements (in ppmv or % v/v), and to a closed-chamber system to measure the flux of each gas, i.e., an amount of gas leaking per square meter per day, at ground level.

Individual measurements can be taken quickly, e.g., in 5-10 minutes. Measurement data can be processed locally, e.g., at the gas development site, to identify anomalies of any gas for which the sensor has been configured to detect. The location of each gas sensor 306 can be recorded, e.g., as global positioning system (GPS) coordinates, and used during analysis of the measurements. The gas sensors 306 can be networked, e.g., with each other or with one or more computers. Measurement data from one or more of the gas sensors 306 can be communicated to one or more computers, e.g., using wireless, cellular, Bluetooth, or wired communication, for data collection, analysis, and/or visualization.

In some implementations, with active shale-gas production, possible gas leaks on the surface can also be rapidly detected by a handheld methane laser sensor, based on infrared adsorption spectroscopy; these leaks will be target points for installing the methane flux sensors. The gas sensors 306 can be then positioned based on the handheld methane sensor results

Locations for positioning the gas sensors 306 can be selected based on the nature of the geographic region, the position of well and wellbore, as well as other factors. For example, the position of the gas sensors 306 can be established according to a specified pattern relative to the gas well 302 or relative to the particular topology of the geographic area 301.

The gas sensors 306 can be used manually, for quick spatial surveys, or can be positioned in fixed automatic stations for long-term monitoring, prior to gas development as well as during and after hydraulic fracturing procedures. For example, while remaining in position, the gas sensors 306 can periodically obtain measurements, e.g., once a day, once a week, etc. The gas sensors 306 can be positioned for measurement over a short sampling time. For example, one or more gas sensors 306 can be used to sample gas measurements from each of multiple locations and then removed. In some other implementations, the gas sensors 306 can be left in position for long-term monitoring of the geographic region surrounding the gas well 302.

FIG. 4 is a flow diagram of an example process 400 for detecting gas leakage. Surface gas sensor measurements are obtained (405). Surface gas sensor measurements can be obtained, for example, from one or more gas sensor devices positioned within a geographic area relative to a gas well site. For example, a number of methane flux sensors or methane concentration sensors, as described above, can be positioned in the geographic area. The respective locations of the sensors can be according to a specified pattern or in response to initial sensor measurements, e.g., handheld laser sensor measurements to identify target locations. In particular, the gas sensor devices can be positioned at ground level and do not require digging individual wells beneath the surface in order to conduct measurements.

Each gas sensor can be configured to perform sensor measurements according to a specified schedule, in response to manual initiation, or by computer control. For example, sensors can be positioned for a single measurement or for long term monitoring. During long term monitoring, a measurement schedule can be used to periodically take gas sensor measurements. Alternatively, in the single use case, a user can initiate each measurement manually or by computer after the sensor is positioned.

The obtained surface gas sensor measurements are evaluated to determine whether gas is detected (410). In some cases, the detection of gas is determined with reference to a specified threshold value. For example, when baseline measurements are performed prior to shale-gas production, any natural seepage detected can be used to establish a baseline threshold.

In particular, for each sensor measurement, it can be determined whether detected levels of gas exceed a specified threshold concentration. The threshold value for methane may simply be equivalent to the average atmospheric concentration, e.g., ˜2 ppmv, when the measurement refers to gas concentration in the soil, or may be zero when the measurement refers to the gas flux to the atmosphere, expressed in grams of CH4 per square meter per day. In particular, in normal condition, methane flux in dry soil is typically negative or nil, due to methanotrophic bacteria activity in the soil. Therefore, a positive methane flux value is an indication of an underground gas source. The threshold can be zero (both in terms of concentrations and fluxes) for all other hydrocarbons and BTEX. If no gas is detected or no gas is detected beyond a threshold established by baseline measurements, no further action need be taken with respect to that sensor during the current measurement process.

For each gas sensor measurement in which gas is detected, further analysis is performed to determine provenance (415). In particular, for the determination of the origin of methane, isotopic analysis can be performed. Isotopic analysis can be used to distinguish between thermogenic methane originating from gas development and methane having, for example, shallow microbial origin unrelated to gas development. Additionally, gases from shale development areas typically have well-defined isotopic compositions that apply to the particular shale development. For specific identification of the gas origin, isotope analyses from the surface can be compared to hydraulic fracturing backflow gases form the well site in question, which would allow a correlation between surface gas and well gas. Thus, the source of the methane can be determined.

In some implementations, particular gas sensors, e.g., sensors 306, are associated with an isotopic analyzer for locally performing the analysis to determine the methane origin. In some alternative implementations, samples from the gas measurements can be extracted and analyzed in another location eight locally or at a laboratory.

A geographic extent of gas leakage can be estimated based on the determination (420). For example, based on the locations of gas sensor devices that had methane measurements indicating thermogenic origin, an estimated geographic region of methane leakage can be determined. The locations of the gas sensor devices, boundaries of the geographic region, and measured methane concentrations can be recorded. This recorded data can be used for further analysis and processing.

The further processing can include optionally generating a visual representation of the gas leakage data (425). The visual representation can include a map plot that indicates one or more of the estimated geographic extent, gas sensor locations, and/or concentration amounts. In some implementations, a map of the geographic area surrounding the gas well is augmented with the data to indicate the geographic extent of the gas leakage. In some other implementations, a three-dimensional or surface plot is generated that illustrates methane concentration based on location, e.g., longitude and latitude coordinates. Alternatively, a scatter plot of measurement locations and concentrations can be generated with respect to location coordinates, e.g., longitude and latitude.

In some implementations, lithologic data including rock properties such as permeability and porosity are collected from the vertical part of the wellbore 104. This data can be used as input into a computer model that simulates migration of methane in order to estimate the time required for the movement of gas through the rocks between horizontal well and the surface. This information can be used to determine the optimal time for a gas leakage survey. For example, if a first set of measurements immediately after hydraulic fracturing do not detect methane leakage, data indicating that any released methane leaks would not reach the surface for four weeks would provide an indication of when follow up measurements should occur. Thus, time lapse measurement can be used to more accurately determine whether or not gas leakage is occurring by taking into account the time necessary for the gas molecules to migrate to the surface.

A determination is made as to whether or not to perform groundwater contamination analysis (430). Groundwater analysis, particularly for hydrocarbon components, is optionally performed within the geographic region of gas leakage to determine whether it is likely that groundwater contamination has occurred. If groundwater analysis is not performed (no branch), the process ends. Optionally, additional gas monitoring can be performed (432). For example, periodic measurements as part of a long term monitoring in which the process repeats.

If groundwater analysis is determined to be performed (yes branch), additional measurement data within the identified geographic region of gas leakage is obtained (435). In some implementations, additional gas sampling is performed within the geographic region using the same gas sensor devices or additional gas sensors. Groundwater data can be obtained and analyzed using underground sampling including directly in the water table. Alternatively, groundwater data can be obtained from surface water sources including springs, ponds, etc.

The obtained sensor measurements are analyzed for hydrocarbon contamination (440). In particular, gas analysis of methane concentrations as well as analysis of particular volatile organic compounds can be used to determine groundwater contamination levels. The volatile organic compounds can include benzene, toluene, ethylbenzene, and xylenes, collectively referred to as BTEX. BTEX can derive from escaping gases or fluids from hydraulic fracturing.

Measuring concentrations of these hydrocarbons in ground water can be performed by a gas sensor using Fourier Transform infrared spectrometer. The resulting measurements can be processed to determine a corresponding groundwater concentration. The groundwater concentration can then be compared to threshold levels indicating contamination. For example, particular hydrocarbons can be associated with established toxic concentration levels. For example, concentrations of BTEX hydrocarbons in uncontaminated ground water may range below the detection limit, e.g., <0.5 parts per billion, whereas contaminations from polluting fluids can increase the measured BTEX concentrations to 100 to 120 parts per million. In some implementations, BTEX provenance is determined through isotopic analysis. Hydrocarbon contamination can be considered to occur only when the origin of BTEX is determined to be from gas development.

The geographic extent of groundwater contamination can be determined and results provided (445). For example, based on the locations of the hydrocarbon measurements that exceed specified threshold values, an estimated geographic region of hydrocarbon groundwater contamination can be determined. The locations of the gas sensor devices, boundaries of the geographic region, and measured hydrocarbon concentrations, e.g., for each of the respective BTEX components, can be recorded. This recorded data can be used for further analysis and processing.

Groundwater contamination data can be provided. In some implementations, the data can be used to generate a visual representation of groundwater contamination. The visual representation can include, for example, a map plot that indicates one or more of the estimated geographic extent, sensor locations, and/or concentration amounts. In some implementations, a map of the geographic area surrounding the gas well is augmented with the data to indicate the geographic extent of the groundwater contamination. In some other implementations, a three-dimensional plot is generated that illustrates groundwater contamination concentrations based on location. Alternatively, a scatter plot of measurement locations and concentrations can be generated with respect to location coordinates, e.g., longitude and latitude.

In some implementations, only gas leakage steps are performed, e.g., separately from any groundwater analysis. In some other implementations, gas leakage and groundwater analysis is performed. Furthermore, in some alternative implementations, groundwater analysis is performed alone or in conjunction with concurrent methane measurements.

FIG. 5 is a diagram 500 of an example area of contamination 502 relative to gas sensor locations. In particular, diagram 500 shows a geographic area relative to a gas well 504. Gas sensor locations are represented by circles 506 positioned in the geographic area. Additionally, each gas sensor includes an indicator 508 of the concentration of sensor measurements at that location. For example, for methane gas leakage, each vertical bar indicates the concentration of methane having thermogenic origin detected by the corresponding gas sensor 506. The area of contamination 502 illustrates a geographic region identified as having contamination. For example, for methane leakage, the geographic region 502 indicates an extent to which the methane leakage was detected based on gas sensor measurements. Similarly, the area of contamination 502 can indicate a geographic region of groundwater contamination in which BTEX or other hydrocarbon contamination exceeds threshold values.

FIG. 6 is a three-dimensional plot 600 showing an example of methane gas concentration with respect to geographic location. In particular, the plot 600 illustrates a degree of methane flux with respect to location. For example, when plotting methane leakage relative to geographic locations, a particular coordinate system can be used. In this example shown by FIG. 6, geographic location is shown by longitude and latitude values. Other coordinate systems can be used. The height of the methane flux represents the respective concentration of methane leakage measured at that location. Thus, the three-dimensional plot 600 provides a three dimensional view of methane leakage amounts over a given geographic area. Additionally, in some implementations, coloring or shading can be used to differentiate between different concentration levels. In some implementations, the three-dimensional plot 600 can represent hydrocarbon concentrations. Additionally, the three-dimensional plot 600 can include additional features, for example, labels indicating the location of one or more gas wells as well as the horizontal wellbore paths.

In some implementations, isotope analyses can be visualized in a similar fashion as methane concentrations and would allow an identification of the origin of methane in high concentration areas. The isotope analyses data can be compared to gas samples from particular wells to prove or disprove the gas development as the origin of the contamination.

While the above description has generally been made with respect to the example of natural gas production, the techniques can be applied to other applications. For example, the sensors can be positioned around old or capped oil wells to detect gas leakage from leaking cementation of the well. Similarly, the sensors can be positioned along underground pipelines or underground gas storage facilities to detect leaks.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory program carrier for execution by, or to control the operation of, data processing apparatus. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “data processing apparatus” encompasses all kinds of apparatus, devices, and machines for processing data, including by way of example a programmable processor, a computer, or multiple processors or computers. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, a module, a software module, a script, or code) can be written in any form of programming language, including compiled or interpreted languages, or declarative or procedural languages, and it can be deployed in any form, including as a stand-alone program or as a module, component, subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network.

In some implementations, the computer code specifically employs statistical analysis of data for a comparison of background/baseline data before hydraulic fracturing with measurements after hydraulic fracturing to determine a statistically significant change of the methane flux. Additionally, in some further implementations, the computer code is configured to model gas migration, e.g., through rock or other geologic structures.

Computers suitable for the execution of a computer program include, by way of example, can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A method comprising:
obtaining surface gas sensor measurements from each of a plurality of locations within a geographic area prior to shale-gas development;
determining whether natural gas seepage is occurring and establishing a corresponding baseline threshold value;
obtaining additional surface gas sensor measurements after shale-gas development;
determining that the additional gas sensor measurements for one or more of the plurality of locations exceed the baseline threshold level;
performing field analysis of the gas sensor measurements that were determined to exceed the specified threshold to determine gas provenance, wherein the analysis distinguishes between natural gas seepage and thermogenic leakage; and
presenting data indicating a geographic extent of gas thermogenic leakage based on the analysis.

2. The method of claim 1, comprising:
obtaining isotope data for methane identified in the analysis as having thermogenic provenance.

3. The method of claim 2, comprising:
comparing the obtained isotope data with additional isotope data obtained from gases removed during hydraulic fracturing of a horizontal well.

4. The method of claim 2, further comprising:
obtaining groundwater samples at locations corresponding to locations identified as having thermogenic provenance; and
analyzing the groundwater for evidence of hydrocarbon contamination.

5. The method of claim 2, comprising:
providing data visualizing a geographic extent of hydrocarbon contamination.

6. The method of claim 3, wherein visualizing the geographic extent includes generating one or more of a map indicating a geographic region of hydrocarbon contamination, a three-dimensional plot of hydrocarbon concentration with respect to location coordinates, or a scatterplot of hydrocarbon concentration with respect to location coordinates.

7. The method of claim 2, wherein analyzing the groundwater data includes measuring levels of BTEX.

8. The method of claim 1, wherein presenting data indicating a geographical extent of gas leakage includes generating a map representation indicating the geographical extent of the gas leakage.

9. The method of claim 6, wherein generating the map representation includes generating a three-dimensional plot indicating a concentration of gas leakage with respect to geographic location.

10. The method of claim 6, wherein generating the map representation includes generating a scatter plot indicating a concentration of gas leakage with respect to geographic location.

11. The method of claim 1, wherein obtaining gas sensor measurements includes receiving data collected from a plurality of gas sensors, each gas sensor being positioned at a particular location within the geographic region.

12. The method of claim 9, wherein the gas sensors include one or more of a gas flux meter, a laser sensor, or an infrared Fourier transform infrared spectrometer.

13. The method of claim 1, wherein determining gas origin includes performing isotope analysis.

14. The method of claim 1, wherein the data is presented relative to hydraulic fracturing operations.

15. The method of claim 1, further comprising:
determining whether to perform hydrocarbon analysis;
obtaining hydrocarbon data from groundwater samples associated with locations identified in the analysis as having a gas measurements of thermogenic provenance that exceed the threshold concentration; and
performing isotopic analysis of the hydrocarbon data for evidence of hydrocarbon contamination.

16. A system comprising:
a plurality of surface gas sensors, each surface gas sensor configure to collect data associated with measurements of one or more of methane or particular hydrocarbon concentrations;
one or more computers configured to perform operations on the data from the plurality of gas sensors to determine a geographic extent of gas contamination and to generate visual representations of the data.

17. A method comprising:
obtaining gas sensor measurements from each of a plurality of locations within a geographic area, wherein the gas sensors are surface sensors measuring gas emitted from the surface;
determining that the gas sensor measurements for one or more of the plurality of locations indicates gas leakage;
analyzing groundwater samples associated with locations in which the gas sensor measurements indicated gas leakage; and
presenting data indicating a geographic extent of hydrocarbon contamination based on the analysis.
